(19) United States
(12) Patent Application Publication
(10) Pub. No.: US 2016/0175350 A1
(43) Pub. Date: Jun. 23, 2016

(54) ALUMINUM CHLOROHYDRATE SALTS EXHIBITING HIGH SEC PEAK 3

(71) Applicant: COLGATE-PALMOLIVE COMPANY, New York, NY (US)

(72) Inventors: Viktor Dubovoy, Cresskill, NJ (US); Long Pan, Cherry Hill, NJ (US); Scott Smart, Piscataway, NJ (US); Kristen Reale, Blairstown, NJ (US)

(73) Assignee: Colgate-Palmolive Company, New York, NY (US)

(21) Appl. No.: 14/909,102

(22) PCT Filed: Jul. 31, 2013
(86) PCT No.: PCT/US13/52845

§ 371 Date: Jan. 30, 2016

(51) Int. Cl. A61K 33/08; A61K 8/26; C02F 1/52; C01F 7/56; C01G 25/00; C07F 5/06; A61Q 15/00

(52) U.S. Cl. CPC A61K 33/08; C07F 5/069; A61K 8/26; A61Q 15/00; C01F 7/56; C01G 25/006; C02F 1/5245; A61K 2800/75; A61K 2800/58

(57) ABSTRACT

An aluminum chlorohydrate salt having a Peak 3:Peak 4 ratio of at least 10:1 and an amount of Peak 3 material relative based on a total of Peaks 2, 3, 4, and 5 is at least 80% as measured by size exclusion chromatography, together with water treatment compositions, antiperspirant compositions, and oral care compositions, comprising the same, and methods for making and using the same.

### BACKGROUND

Aluminium chlorohydrate is an aluminum salt formed from aluminum or aluminum hydroxide, hydrochloric acid, and water, and optionally also including zirconium and/or complexing agents such as amino acids or polyols. Such salts are used in deodorants and antiperspirants, and as coagulants or flocculants in water purification processes. In aqueous solution, these salts form complex substructures, e.g., Al₁₃, units with a Keggin ion structure, which in turn form larger polymeric species with molecular weights (MW) of over 1000 Daltons. The precise ratios of elements in these salts and the precise three dimensional structures formed can be controlled by method of manufacture. Typically, aluminum chlorohydrate salts may have the general formula AlₙCl₍₃ₙ₋ₘ₎(OH)ₘ, e.g., Al₂Cl(OH)₅ or Al₄Cl₂(OH)₁₀. These salts may additionally be in complex with zirconium and/or an amino acid, ammonium acid, or a polyol, e.g., Al/Zr tetrachlorohydrex-Gly ([Al₄Cl₂(OH)₁₀·ZrOCl₂]NH₂CH₂COOH). Aluminum chlorohydrate salts approved for use as antiperspirants in the United States are listed in 21 CFR 350.10.

Size exclusion chromatography (“SEC”) or gel permeation chromatography (“GPC”) provides information on polymer distribution of aluminum chlorohydrate in aqueous solutions. For antiperspirant salts generally, including aluminum chlorohydrate, aluminum/zirconium chlorohydrate, and complexes thereof, distinctive peaks have been identified, corresponding to different size populations of the polymer complexes in solution, appearing in a chromatogram as peaks 1, 2, 3, 4 and a peak known as “5,6”. Peak 1 is the larger Zr species (greater than 60 Angstroms), and is not present in salts without zirconium. Peaks 2 and 3 are larger aluminum species. Peak 4 is a smaller aluminum species (aluminum oligomers, or small aluminum cluster) and has been correlated with enhanced efficacy for both Al and Al/Zr salts. Peak 5, 6 is the smallest aluminum species.

Alumunum chlorohydrate salts used in commercial antiperspirant formulations are typically activated or enhanced to contain large amounts of Peak 4 species. Commonly, such salts further comprise zirconium and glycine, and are sometimes referred to as zirconium-aluminum chlorohydrex glycine (“ZAG” or “AZG”). There remains a need, however, for salts which are less irritating to the skin and less damaging to fabric when used in antiperspirant formulations, and also for salts which have enhanced flocculation properties when used in water purification processes,

### SUMMARY

Provided is aluminum chlorohydrate salts, optionally complex with a complexing agent such as glycine and/or additionally comprising zirconium, which, when measured by size exclusion chromatography in aqueous solution, contain predominantly Peak 3. The invention further provides methods of making and using such salts. The methods for synthesizing Peak 3 enhanced salts include conversion of salts containing high levels of Peak 4, or 5/6 (e.g. AlCl₃) by thermal treatment to provide a salt which contains predominantly Peak 3 species. For convenience, these salts are sometimes referred to herein as AC113.

In one embodiment, provided is ACH3 having a Peak 3/Peak 4 ratio of at least 10:1 and an amount of Peak 3 material relative based on a total of Peaks 2, 3, 4, and 5 is at least 80% as measured by size exclusion chromatography. The ACH3 may optionally further comprise (i) zirconium and/or (ii) one or more complexing agents selected from a) amino acids, e.g., glycine, b) ammonium acids, e.g., betaine, c) polyols, e.g., diols, for example propylene glycol or polyethylene glycol. d) carboxylic acids, e) hydroxyl acids, and f) sulfonic acids.

In another embodiment, the invention provides a method of making the ACH3 comprising thermal treatment of an aluminum chlorohydrate salt which is substantially free of calcium ions, e.g., containing less than 1 percent calcium ion. For example, the ACH3 active can be produced by refluxing a reaction mixture containing sufficient amount of ACH to have preferably at least 3% aluminum (preferably above 6%) in the presence of a previously mentioned complexing agent (e.g. glycine) in the absence of Ca² ion. The Peak 3 active of the invention can also be produced by refluxing purified Peak 4 after Ca²⁺ ion is removed. Prior art commercial production processes do not result in material with Peak 3 levels of at least 90% relative to peak 4, and moreover differ e.g., in that ACH for water-treatment applications do not include complexing agents such as glycine, while prior art commercial production processes of ACH for antiperspirants typically contain Ca²⁺ or are otherwise manipulated to result in high levels of Peak 4.

The ACH3 is found to be particularly useful for water treatment, exhibiting superior flocculating capability. The invention thus provides in one embodiment compositions and methods for water treatment.

The ACH3 is also useful for antiperspirant formulations. Therefore, in another embodiment the ACH3 provides compositions and methods for reducing perspiration and odor, particularly underarm sweat and odor.

We have moreover discovered that the Peak 3 species are of an optimal size, charge and stability to provide adequate dentinal microtubule occlusion, and the ACH3 is therefore useful in oral care formulations to treat and reduce dental hypersensitivity and erosion. Thus in yet another embodiment, the invention provides compositions and methods for oral care, particularly to treat and reduce dental hypersensitivity and erosion.

Further areas of applicability of the present invention will become apparent from the detailed description and examples provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses. As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls. Unless otherwise specified, all percentages and amounts expressed herein and elsewhere in the specification should be understood to refer to percentages by weight. The amounts given are based on the active weight of the material.

As used throughout, ranges are used as a shorthand for describing each and every value that is within the range. Any value within the range can be selected as the terminus of the range.

In a first embodiment, the invention provides an aluminum chlorohydrate salt having a Peak 3:Peak 4 ratio of at least 10:1 and an amount of Peak 3 material relative based on a total of Peaks 2, 3, 4, and 5 is at least 80% as measured by size exclusion chromatography (“Composition 1”).
1.1. Composition 1 wherein a Peak 3:Peak 4 ratio is at least 15:1, optionally 20:1.
1.2. Composition 1 or 1.1 wherein the Peak 3:Peak 2 ratio is at least 10:1, optionally 15:1 or 20:1.
1.3. Any of the foregoing compositions wherein the amount of Peak 3 material relative based on the total of Peaks 2, 3, 4, and 5 is at least 90%, optionally at least 95% or 100%.
1.4. Any of the foregoing compositions having a Peak 3:Peak 4 ratio of at least 10:1, when measured in an aqueous solution, e.g. 8% aluminum aqueous solution, as measured by size exclusion chromatography
1.5. Any of the foregoing compositions wherein the Peak 3:Peak 4 ratio is at least 20:1.
1.6. Any of the foregoing compositions wherein the Peak 3:(Peak 4+Peak 2) ratio is at least 10:1, e.g. at least 15:1, e.g., at least 20:1.
1.7. Any of the foregoing compositions wherein the composition further comprises one or more complexing agents selected from a) amino acids [e.g., glycine], b) ammonium acids [e.g., betaine (trimethyiglycine)], c) polyols [e.g., diols, for example propylene glycol or polyethyleneglycol], d) carboxylic acids, e) hydroxyl acid, and f) sulfonic acids, and optionally a molar ratio of complexing agent to aluminum in certain embodiments can be not greater than 3:1, e.g. 0.01:1 to 3:1, 0.1:1 to 3:1, 0.5:1 to 2:1, 1:1 to 1.5:1, or about 1:1.
1.8. Any of the foregoing compositions wherein the composition comprises an amino acid.
1.9. Any of the foregoing compositions wherein the composition comprises glycine.
1.10. Any of the foregoing compositions wherein the composition further comprises zirconium.
1.11. Any of the foregoing compositions wherein the aluminum chlorohydrate has the general formula AlₙCl₍₃ₙ₋ₘ₎(OH)ₘ wherein n and m are integers.
1.12. Any of the foregoing compositions wherein the composition comprises glycine in an glycine:aluminum molar ratio of 0.1:1 to 3:1, e.g., 1.5:1 to 1:1.5, e.g., about 1:1.
1.13. Any of the foregoing compositions which is substantially free of calcium ion, e.g., less than 1% calcium ion.
1.14. Any of the foregoing compositions having an aluminum:chloride molar ratio of 0.3:1 to 3:1, e.g., about 2:1.
1.15. Any of the foregoing compositions comprising zirconium having a molar ratio of Al:Zr from 5:1 to 10:1, e.g., about 8:1.
1.16. Any of the foregoing compositions wherein the aluminum chlorohydrate is comprised predominantly of octahedral coordinated aluminum atoms, e.g., having a ratio of octahedral:tetrahedral configuration of at least 15:1, e.g., at least 20:1, e.g., substantially all octahedral configuration, for example as determined by ²⁷Al NMR spectroscopy showing a dominant peak from 0-59 ppm, e.g. at about I 11 ppm. (corresponding to octahedral Al) which is larger, e.g., at least 5×, e.g., at least 10× or at least 20× larger, than the peak at 60-85 ppm, e.g. at about 63.5 ppm (corresponding to tetrahedral Al).
1.17. Any of the foregoing compositions wherein the aluminum chlorohydrate is selected from complexed or uncomplexed aluminum chlorohydrate, aluminum chlorohydrex polyethylene glycol, aluminum chlorohydrex propylene glycol, complexed or uncomptexed aluminum dichlorohydrate, aluminum dichlorohydrex polyethylene glycol, aluminum diehlorohydrex propylene glycol, complexed or uncomplexed aluminum sesquichlorohydrate, aluminum sesquichlorohydrex polyethylene glycol, aluminum sesquichlorohydrex propylene glycol, complexed or uncomplexed aluminum zirconium octachlorohydrate, aluminum zirconium octachlorohydrex glycine, aluminum zirconium pentachlorohydrate, aluminum zirconium pentachlorohydrex glycine, complexed or uncomptexed aluminum zirconium tetrachlorohydrate, aluminum zirconium tetrachlorohydrex glycine, complexed or uncomplexed aluminum zirconium trichlorohydrate, and aluminum zirconium triehlorohydrex glycine.
1.18. Any of the foregoing compositions when made by heating an initial aluminum salt solution until the Peak 3:Peak 4 ratio is at least 10:1 and an amount of Peak 3 material relative based on a total of Peaks 2, 3, 4, and 5 is at least 80% as measured by size exclusion chromatography, wherein the aluminum salt is at least one of aluminum chloride, aluminum chlorohydrate, aluminum chlorohydrex polyethylene glycol, aluminum chlorohydrex propylene glycol, aluminum dichlorohydrate, aluminum dichlorohydrex polyethylene glycol, aluminum dichlorohydrex propylene glycol, aluminum sesquichlorohydrate, aluminum sesquichlorohydrex polyethylene glycol, aluminum sesquichlorohydrex propylene glycol, aluminum zirconium octachlorohydrate, aluminum zirconium octachlorohydrex glycine, aluminum zirconium pentachlorohydrate, aluminum zirconium pentachlorohydrex glycine, aluminum zirconium tetrachlorohydrate, aluminum zirconium tetrachlorohydrex glycine, aluminum zirconium trichlorohydrate, and aluminum zirconium trichlorohydrex glycine.
1.19. Any of the foregoing compositions wherein the aluminum chlorohydrate salt is in combination or association with a substantially anhydrous carrier, e.g., a carrier having less than 10%, preferably less than 5% water by weight of the total composition.
1.20. Any of the foregoing Compositions 1-1.21 for use as, or in the manufacture of, a flocculant, e.g., for treating and purifying water.
1.21. Any of the foregoing Compositions 1-1.21 for use as, or in the in the manufacture of an antiperspirant.
1.22. Any of the foregoing Compositions 1-1.21 for use as, or in the manufacture of an oral care product, e.g., to treat and/or reduce dental hypersensitivity and/or erosion.
1.23. Any of the foregoing Compositions when made by heating an initial ACH solution until Peak 3 becomes the dominant species, e.g., when made according to any of Synthesis 1, et seq. infra.

The relative peak values may be determined using size exclusion chromatography (SEC). The relative retention time (“Kd”) for each of the peaks varies depending on the experimental conditions, but the peaks remain relative to each other. Data for Tables in the examples is obtained using an SEC chromatogram using the following parameters: Waters® 600 analytical pump and controller. Rheodyne® 77251 injector, Protein-Pak® 125 (Waters) column, Waters 2414 Refractive index Detector. 0.1% potassium nitrate (w/v) with 0.055% nitric acid (w/v) mobile phase, 1 ml/min flow rate, 2.0 microliter injection volume. Data is analyzed using Water® Empower software (Waters Corporation, Milford, Mass.). The concentration of the antiperspirant in solution does not affect the retention time in the instrument.

The design of modern AP salts generaly aims at actives with high levels of low molecular weight Al and Zr species, which is reflected in a SEC chromatogram that has intense Peak 4 and low Peaks 1, 2, and 3, in contrast to the present invention which aims at compositions having relatively high Peak 3 content. Throughout the present study, the relative concentration of Peaks 1-5 are estimated based on the following SEC peak area ratios (or percentages):

$${f}_{Pi} = \frac{Pi}{Σ Pj} i = 1 , 2 , 3 , 4 , 5 ; j = 2 , 3 , 4 , 5$$

where fPi is the fraction of peak i, and Pi or Pj represent the intensity of peaks Pi or Pj, respectively, intensity generally correlating with area under the curve or amount of material. As noted above, Peak 1 is a zirconium peak and is not present in a zirconium-free salts, so the sum of Pj reflects the total amount of aluminum chlorohydrate species, In brief, a preferred aluminum chlorohydrate salt would have a very low fP1, fP2, fP4, and/or fP5, and a high fP3.

A variety of hydrolytic Al species exist and it is possible to distinguish large aqueous aluminum hydroxide molecules using spectroscopic methods such as ²⁷Al NMR which elucidates the structural environment surrounding Al atoms which are embodied in various forms. There are typically two regions in a ²⁷Al NMR spectrum, one of Al nuclei that are octahedrally coordinated (0 ppm-60 ppm and the other of Al nuclei that are tetrahedrally coordinated (60 ppm-85 ppm). The octahedral region is exemplified by the hexa-aqua Al species, i.e. monomeric Al, which resonates sharply near 0 ppm. The tetrahedral region is exemplified by resonance near 63.5 ppm from the Al₁₃ polyhydroxyoxoaluminum cation. Al₁₃ is composed of 12 octahedrally coordinated Al atoms surrounded by one centrally-cited Al atom which is tetrahedrally coordinated. The Al₃₀ polyhydroxyoxoaluminum cation is essentially a dimer of the Al₁₃polyhydroxyoxoaluminum cation and contains 2 tetrahedrally sited Al atoms which yield a somewhat broad resonance near 70 ppm. Depending on calibration, the above ppm values can vary. The values for these peaks are approximately where the resonance occurs.

Peak 3 is found to be predominantly made up of octahedrally coordinated Al species, showing a dominant peak at about 11 ppm (corresponding to octahedral Al) which is much larger than the peak at about 70 ppm (corresponding to tetrahedral Al).

The compositions may be made in a variety of ways involving a stepwise procedure to neutralize aluminum chloride in solution (optionally buffered) using inorganic basic salts. The procedure generally includes the step of heating an aqueous solution containing an aluminum chloride compound (optionally with a buffer agent) at a temperature of about 50° C. to about 95° C. to reflux for a period of time of about 1 hour to about 5 hours. In one such embodiment, an aqueous solution containing an aluminum chloride compound is heated at a temperature of about 75° C. to about 95° C. to reflux for a period of time of about 3 hours to about 4 hours. In another such embodiment, an aqueous solution containing an aluminum chloride compound and a buffer agent is heated at a temperature of about 75° C. to about 95° C. to reflux for a period of time of about 3 hours to about 4 hours. In one embodiment, the temperature is about 85° C. Optionally a complexing agent as described above may be added. To adjust the pH of the aluminum salt solution, an aqueous solution of an inorganic base is added to the heated solution to thereby obtain a pH adjusted aluminum salt solution having a hydroxide to aluminum molar ratio of about 1:1 to about 4:1, and a pH of about 2 to about 5. In one such embodiment, the hydroxide to aluminum molar ratio of about 2:1 to about 3:1. In another such embodiment, the hydroxide to aluminum molar ratio is about 2.1:1 to about 2.6:1. In some embodiments, a zirconium salt may also be added to the pH adjusted aluminum salt solution. In one other such embodiment, the molar ratio of Al:Zr is about 5:1 to about 10:1.

In one embodiment, an aqueous aluminum chloride salt solution is buffered with betaine monohydrate and held at about 50° C. to about 95° C. to reflux for a period time of about 1 to about 6 hours. To the heated solution, an aqueous solution of an inorganic base is added dropwise over a period of time of about 1 to about 3 hours while maintaining the aluminum-betaine solution at about 50° C. to about 95° C. to reflux,

In one embodiment, an aqueous solution containing an aluminum chloride compound is buffered with betaine monohydrate and held at about 75° C. to about 95° C. to reflux for a period of time of about 3 hours to about 4 hours. In another such embodiment, an aqueous solution of an inorganic base is added dropwise over a period of time of about 1 to about 3 hours while maintaining the aluminum-betaine solution at about 75° C. to about 95° C. to reflux. In another embodiment, an aqueous solution of an inorganic base is added over a period of time in a series of additions while maintaining the aluminum-betaine solution at about 75° C. to about 95° C. to reflux. In one such embodiment, the inorganic base is added in at least 3 additions. In another such embodiment, the inorganic base is added in at least 5 additions. In another embodiment, a ZrOCl₂ solution is added to the pH adjusted aluminum-betaine solution. In one such embodiment, the molar ratio of Al:Zr is about 8. In another such embodiment, the molar ratio of Al:Zr is about 7. In one other such embodiment, the molar ratio of Al:Zr is about 9.

In another embodiment, an aqueous aluminum chloride solution is buffered with glycine and held at about 50° C. to about 95° C. to reflux for a period time of about 1 to about 6 hours. To the heated solution, an aqueous solution of an inorganic base is added dropwise over a period of time of about 1 to about 3 hours while maintaining the aluminum-glycine solution at about 50° C. to about 95° C. to reflux. In one such embodiment, the solution has an aluminum to glycine molar ratio of about 0.1. In another such embodiment, the solution has an aluminum to glycine molar ratio of about 1.

In another embodiment, a ZrOCl₂ solution is added to the pH adjusted aluminum-glycine solution. In one such embodiment, the molar ratio of Al:Zr is about 8. In another such embodiment, the molar ratio of Al:Zr is about 7. In one other such embodiment, the molar ratio of Al:Zr is about 9.

For the above methods, the aluminum chloride salt and inorganic base may be obtained from a variety of sources. In one embodiment, the aluminum chloride salt includes aluminum trichloride, aluminum chlorohexahydrate and aluminum dichlorohydrate. In one such embodiment, the aluminum chloride salt is aluminum chlorohexahydrate.

In one embodiment, the inorganic base can be at least one base chosen from metal hydroxides, calcium hydroxide, strontium hydroxide, sodium hydroxide, barium hydroxide, metal oxides, calcium oxide, strontium oxide, and barium oxide.

The polymerization of the antiperspirant actives in aqueous solutions and the correspondent gelation process are followed by monitoring the molecular weight profile of the polyoxohalides in time by SEC. The relative retention time (“Kd”) for each of these peaks varies depending on the experimental conditions, but the peaks remain relative to each other. The concentration of the antiperspirant in solution does not affect the retention time in the machine.

In one embodiment, the ACH3 is made using commercial enhanced ACH, a partially neutralized polyaluminum chloride system composed of Al clusters that elute primarily under SEC peak 3 and 4 with small amounts of peak 5.

The above syntheses, however, are not specific for high Peak 3 concentration. Peak 3 levels may be monitored and may be enhanced by thermal treatment of relatively concentrated ACH solution, optionally in presence of complexing agent, e.g., glycine, and/or by thermal treatment of solution with high Peak 4 levels, to provide enhanced levels of Peak 3 material.

The present invention thus provides for a method (Synthesis 1) of making an aluminum chlorohydrate salt having a Peak 3:Peak 4 ratio of at least 10:1 and an amount of Peak 3 material relative based on a total of Peaks 2, 3, 4, and 5 is at least 80% as measured by size exclusion chromatography, e.g., any of Composition 1 et seq., comprising heating an initial ACH solution at a temperature of 40-80° C., e.g. 50-55° C. e.g., about 55° C., optionally in presence of complexing agent, e.g., glycine, until Peak 3 material becomes the dominant species.
1.1. Synthesis 1 wherein the concentration of the initial ACH solution is 3-20% by weight of aluminum to total solution.
1.2. Synthesis 1 or 1.1 wherein the heating is carried out for at least two hours, e.g., 2-60 hrs, e.g., 25-30 hours.
1.3. Any of the foregoing syntheses, wherein glycine is present at a molar ratio of 0.5-1.5:1, e.g., about 1:1, of Al:Glycine.
1.4. Any of the foregoing syntheses wherein the initial ACH solution is at least 90% Peak 4.
1.5. Any of the foregoing syntheses further comprising diluting the product of any of the foregoing syntheses, e.g., by 25-75%, e.g., about 50%, and heating further at a temperature of 40-80° C., e.g. 50-55° C.
1.6. Any of the foregoing syntheses comprising heating an initial aluminum salt solution until the Peak 3:Peak 4 ratio is at least 10:1 and an amount of Peak 3 material relative based on a total of Peaks 2, 3, 4, and 5 is at least 80% as measured by size exclusion chromatography, wherein the aluminum salt is at least one of aluminum chloride, aluminum chlorohydrate, aluminum chlorohydrex polyethylene aluminum chlorohydrex propylene glycol, aluminum dichlorohydrate, aluminum dichlorohydrex polyethylene glycol, aluminum dichlorohydrex propylene glycol, aluminum sesquichlorohydrate, aluminum sesquichlorohydrex polyethylene glycol, aluminum sesquichlorohydrex propylene glycol, aluminum zirconium octachlorohydrate, aluminum zirconium octachlorohydrex glycine, aluminum zirconium pentachlorohydrate, aluminum zirconium pentachlorohydrex glycine, aluminum zirconium tetrachlorohydrate, aluminum zirconium tetrachlorohydrex glycine, aluminum zirconium trichlorohydrate, and aluminum zirconium trichlorohydrex glycine.
1.7. Any of the foregoing syntheses wherein the initial ACH solution is made by
   I) heating an aqueous solution containing an aluminum salt having an aluminum to chloride molar ratio of about 0.3:1 to about 3:1, optionally with a buffer or complexing agent, at a temperature of about 50° C. to about 95° C. to reflux for a period of time of about 1 hour to about 5 hours to obtain an aluminum salt solution;
   II) adding an aqueous solution of an inorganic base to obtain an aluminum salt solution having an OH:Al molar ratio of about 2:1 to about 2.6:1 to obtain a pH adjusted aluminum salt solution having a pH of about 2 to about 5; and
   III) optionally adding an aqueous solution containing a zirconium compound to the pH adjusted aluminum salt solution to thereby obtain an aluminum-zirconium salt solution having a molar ratio of aluminum to zirconium of about 5:1 to about 10:1.

In some embodiments, the aluminum chloride salt of Composition 1, et seq. is made from a salt as described in 21 CFR 350.10, e.g., a salt which meets the aluminum to chloride, aluminum to zirconium, and aluminum plus zirconium to chloride atomic ratios described in the U.S. Pharmacopeia-National Formulary. Exemplary aluminum chlorohydrates, aluminum-zirconium chlorohydrates and complexes thereof include:
(a) Aluminum chloride.
(b) Aluminum chlorohydrate.
(c) Aluminum chlorohydrex polyethylene glycol.
(d) Aluminum chlorohydrex propylene glycol.
(e) Aluminum dichlorohydrate.
(f) Aluminum dichlorohydrex polyethylene glycol.
(g) Aluminum dichlorohydrex propylene glycol.
(h) Aluminum sesquichlorohydrate.
(i) Aluminum sesquichlorohydrex polyethylene glycol.
(j) Aluminum sesquichlorohydrex propylene glycol.
(k) Aluminum zirconium octachlorohydrate.
(l) Aluminum zirconium octachlorohydrex glycine.
(m) Aluminum zirconium pentachlorohydrate.
(n) Aluminum zirconium pentachlorohydrex glycine.
(o) Aluminum zirconium tetrachlorohydrate.
(p) Aluminum zirconium tetrachlorohydrex glycine.
(q) Aluminum zirconium trichlorohydrate.
(r) Aluminum zirconium trichlorohydrex glycine.

### Water Treatment:

In one embodiment, the invention provides a composition for water treatment, e.g., as a flocculant or coagulant, comprising an aluminum chlorohydrate salt having a Peak 3:Peak 4 ratio of at least 10:1 and an amount of Peak 3 material relative based on a total of Peaks 2, 3, 4, and 5 is at least 80% as measured by size exclusion chromatography, comprising any of Composition 1 et seq.

The invention thus provides a method of removing solids from water, e.g., reducing turbidity or cloudiness of water, comprising adding to the water an aluminum chlorohydrate salt having a Peak 3:Peak 4 ratio of at least 10:1 and an amount of Peak 3 material relative based on a total of Peaks 2, 3, 4, and 5 is at least 80% as measured by size exclusion chromatography, e.g., comprising any of Composition 1 et seq., and removing the gel thus formed from the water.

### Antiperspirant:

In another embodiment, the invention provides an antiperspirant composition comprising an aluminum chlorohydrate salt having a Peak 3:Peak 4 ratio of at least 10:1 and an amount of Peak 3 material relative based on a total of Peaks 2, 3, 4, and 5 is at least 80% as measured by size exclusion chromatography, e,g., comprising any of Composition 1 et seq.

The aluminum antiperspirant active compositions and/or aluminum-zirconium antiperspirant active compositions may be used in a variety of antiperspirant products. If the product is used as a solid powder, the size of the particles of antiperspirant active of the invention can be any desired size, and may include conventional sizes such as in the range of 2 to 100 microns, with selected grades having an average particle size of 30-40 microns; finer sized grades having an average particle size distribution of 2-10 microns with an average size of about 7 microns as made by a suitable dry-grinding method; and micronized grades having an average particle size of less than about or equal to 2 microns, or less than about or equal to 1.5 microns.

The compositions of this invention may be used to formulate antiperspirants which are well tolerated by consumers having sensitive skin. Such antiperspirants include solids such as sticks and creams (creams sometimes being included in the term “soft solid”), gels, liquids (such as are suitable for roll-on products), and aerosols. The forms of these products may be suspensions or emulsions. These antiperspirant actives can be used as the antiperspirant active in any antiperspirant composition.

Note that where water is listed it is intended to count the contribution of the water present in the antiperspirant solution as part of the overall water content. Thus, water is sometimes listed as part of the actives solution or sometimes listed separately.

In one embodiment the refractive indices of the external and internal phases are matched within 0.005 to obtain a clear product.

Antiperspirant compositions can be packaged in conventional containers, using conventional techniques. Where a gel, cream or soft-solid cosmetic composition is produced, the composition can be introduced into a dispensing package (for example, conventional packages for gels with glide on applicators, jars where the gel or cream is applied by hand, and newer style packages having a top surface with pores) as conventionally done in the art. Thereafter, the product can be dispensed from the dispensing package as conventionally done in the art, to deposit the active material, for example, on the skin. For sticks, sprays, aerosols and roll-arts the compositions can be placed in a conventional types of container (with the inclusion of propellants in aerosols). This provides good deposition of the active material on the skin.

Compositions can be formulated as clear, translucent or opaque products. A desired feature of the present invention is that a clear, or transparent, cosmetic composition, (for example, a clear or transparent deodorant or antiperspirant composition) can be provided. The term clear or transparent according to the present invention is intended to connote its usual dictionary definition; thus, a clear liquid or gel antiperspirant composition of the present invention allows ready viewing of objects behind it. By contrast, a translucent composition, although allowing light to pass through, causes the light to be scattered so that it will be impossible to see clearly objects behind the translucent composition. An opaque composition does not allow light to pass there through. Within the context of the present invention, a gel or stick is deemed to be transparent or clear if the maximum transmittance of light of any wavelength in the range 400-800 nm through a sample 1 cm thick is at least 35%, or at least 50% The gel or liquid is deemed translucent if the maximum transmittance of such light through the sample is between 2% and less than about 35%. A gel or liquid is deemed opaque if the maximum transmittance of light is less than about 2%. The transmittance can be measured by placing a sample of the aforementioned thickness into a light beam of a spectrophotometer whose working range includes the visible spectrum, such as a Bausch & Lomb Spectronic 88 Spectrophotometer. As to this definition of clear, see European Patent Application Publication No. 291,334 A2. Thus, according to the present invention, there are diffrences between transparent (clear), translucent and opaque compositions.

### Oral Care:

Dentinal hypersensitivity is acute, localized tooth pain in response to physical stimulation of the dentine surface as by thermal (hot or cold) osmotic, tactile combination of thermal, osmotic and tactile stimulation of the exposed dentin. Exposure of the dentine, which is generally due to recession of the gums, or loss of enamel, frequently leads to hypersensitivity. Dentinal tubules open to the surface have a high correlation with dentine hypersensitivity. Dentinal tubules lead from the pulp to the cementum. When the surface cementum of the tooth root is eroded, the dentinal tubules become exposed to the external environment. The exposed dentinal tubules provide a pathway for transmission of fluid flow to the pulpal nerves, the transmission induced by changes in temperature, pressure and ionic gradients. The particles of the aluminum chlorohydrate of the invention are surprisingly found to be of a size and charge which is effective in blocking and adhering to the dentinal tubules, thereby reducing this fluid flow and reducing the sensitivity of hypersensitive teeth.

In one embodiment, the invention provides an oral care product (“OC Product 1), e.g., a dentifrice, comprising an aluminum chlorohydrate salt having a Peak 3:Peak 4 ratio of at least 10:1 and an amount of Peak 3 material relative based on a total of Peaks 2, 3, 4, and 5 is at least 80% as measured by size exclusion chromatography, e.g., any of Composition 1 et seq.
1.1. OC Product 1 in the form of a toothpaste, gel, mouthwash, powder, cream, strip, or gum.
1.2. OC Product 1 or 1,1 comprising an aluminum chlorohydrate salt having a Peak 3:Peak 4 ratio of at least 10:1, as measured by size exclusion chromatography (SEC) (for example, by SEC performed in aqueous solution, e.g. 8% aqueous solution), comprising any of Composition 1 et seq. in an orally acceptable base, e.g., a mouthwash, gel, or dentifrice base.
1.3. Any of the foregoing products wherein the amount of aluminum in the product is 3 to 20%, optionally 3 to 6%, e.g., about 4%, by weight.
1.4. Any of the foregoing products in the form of a dentifrice, e.g., wherein the aluminum chlorohydrate salt is present in an effective amount to fill the dentinal tubules upon application.
1.5. Any of the foregoing products comprising a dentifrice base, wherein the dentifrice base comprises an abrasive, e.g., an effective amount of a silica abrasive, e.g., 10-30%, e.g., about 20%.
1.6. Any of the foregoing products further comprising an effective amount of a fluoride ion source, e.g., providing 500 to 3000 ppm fluoride.
1.7. Any of the foregoing products further comprising an effective amount of fluoride, e.g., wherein the fluoride is a salt selected from stannous fluoride, sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof, for example, comprising an effective amount of sodium monofluorophosphate.
1.8. Any of the foregoing products comprising an effective amount of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these, e.g., in an amount of 1-20%, e.g., 2-8%, e.g., ca. 5%, by weight of the composition.
1.9. Any of the foregoing products comprising buffering agents, e.g., sodium phosphate buffer (e.g., sodium phosphate monobasic and disodium phosphate).
1.10. Any of the foregoing products comprising a humectant, e.g., selected from glycerin, sorbitol, propylene glycol, polyethylene glycol, xylitol, and mixtures thereof, e.g. comprising at least 20%, e.g., 20-40%, e.g., 25-35% glycerin.
1.11. Any of the foregoing products comprising one or more surfactants, e.g., selected from anionic, cationic, zwitterionic, and nonionic surfactants, and mixtures thereof, e.g., comprising an anionic surfactant, e.g., a surfactant selected from sodium lauryl sulfate, sodium ether lauryl sulfate, and mixtures thereof, e.g., in an amount of from about 0.3% to about 4.5% by weight, e.g. 1-2% sodium lauryl sulfate (SLS); and/or a zwitterionic surfactant, for example a betaine surfactant, for example cocamidopropylbetaine, e.g., in an amount of from about 0.1% to about 4.5% by weight, e.g. 0.5-2% cocamidopropylbetaine.
1.12. Any of the foregoing products further comprising a viscosity modifying amount of one or more of polysaccharide gums, for example xanthan gum or carrageenan, silica thickener, and combinations thereof.
1.13. Any of the foregoing products comprising gum strips or fragments.
1.14. Any of the foregoing products further comprising flavoring, fragrance and/or coloring.
1.15. Any of the foregoing products comprising an effective amount of one or more antibacterial agents, for example comprising an antibacterial agent selected from halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, zinc citrate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing; e.g., comprising triclosan or cetylpyridinium chloride.
1.16. Any of the foregoing products comprising an antibacterially effective amount of triclosan, e.g. 0.1 -0.5%, e.g. about 0.1%.
1.17. Any of the foregoing products further comprising a whitening agent, e.g., a selected from the group consisting of peroxides, metal chlorites, perborates, percarbonates, peroxyacids, hypochlorites, and combinations thereof.
1.18. Any of the foregoing products further comprising hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate);
1.19. Any of the foregoing products further comprising an agent that interferes with or prevents bacterial attachment, e.g., solbrol or chitosan.
1.20. Any of the foregoing products further comprising a source of calcium and phosphate selected from (i) calcium-glass complexes, e.g., calcium sodium phosphosilicates, and (ii) calcium-protein complexes, e.g., casein phosphopeptide-amorphous calcium phosphate
1.21. Any of the foregoing products further comprising a soluble calcium salt, e.g., selected from calcium sulfate, calcium chloride, calcium nitrate, calcium acetate, calcium lactate, and combinations thereof.
1.22. Any of the foregoing products further comprising a physiologically or orally acceptable potassium salt, e.g., potassium nitrate or potassium chloride, in an amount effective to reduce dentinal sensitivity.
1.23. Any of the foregoing products further comprising an anionic polymer, e.g., a synthetic anionic polymeric polycarboxylate, e.g., wherein the anionic polymer is selected from 1:4 to 4:1 copolymers of maleic anhydride or acid with another polymerizable ethylenically unsaturated monomer; e.g., wherein the anionic polymer is a methyl vinyl ether/maleic anhydride (PVM/MA) copolymer having an average molecular weight (M.W.) of about 30,000 to about 1,000,000, e.g. about 300,000 to about 800,000, e.g., wherein the anionic polymer is about 1-5%, e.g., about 2%, of the weight of the composition.
1.24. Any of the foregoing products further comprising a breath freshener, fragrance or flavoring.
1.25. Any of the foregoing products, wherein the pH of the composition is approximately neutral, e.g., from pH 6 to pH 8 e.g., about pH 7.
1.26. Any of the foregoing products for use to reduce and inhibit acid erosion of the enamel, clean the teeth, reduce bacterially-generated biofilm and plaque, reduce gingivitis, inhibit tooth decay and formation of cavities, and reduce dentinal hypersensitivity.

Also provided are methods to reduce and inhibit acid erosion of the enamel, clean the teeth, reduce bacterially-generated biofilm and plaque, reduce gingivitis, inhibit tooth decay and formation of cavities, and reduce dentinal hypersensitivity, comprising applying an effective amount of an oral care product of the invention, e.g., any of OC Product 1, et seq. to the teeth. The invention further provides an oral care product of the invention, e.g., any of OC Product 1, et seq. for use in any of these methods.

Also provided is the use of an aluminum chlorohydrate salt having a Peak 3:Peak 4 ratio of at least 10:1 and an amount of Peak 3 material relative based on a total of Peaks 2, 3, 4, and 5 is at least 80% as measured by size exclusion chromatography, e.g., any of Composition 1 et seg. in the manufacture of an oral care product, e.g., any of OC Product 1, et seq., e.g., to reduce and inhibit acid erosion of the enamel, clean the teeth, reduce bacterially-generated biofilin and plaque, reduce gingivitis, inhibit tooth decay and formation of cavities, and reduce dentinal hypersensitivity.

### EXAMPLES

### Example 1

### Making High Peak 3 Aluminum Chlorohydrate

Reach™ 103 aluminum chlorohydrate solutions with Al concentrations 4, 6, 8, 10, 12, 14 and 16% (w/w) are prepared and aged in a 55° C. oven for 2 hours. The 4, 6, 8, 10, 12, 14 and 16% Al samples are diluted to 1% Al prior to SEC analysis. Peak 3 concentration increases linearly with Al concentration during the thermal treatment, so it is seen that a product which is predominantly Peak 3 material (ACH3) can be obtained by thermally treating a relatively concentrated solution of ACH.

TABLE 1
SEC Data of Reach ™ 103 ACH Aging Study (2 hrs. 55° C.)

| Amount Al (wt. %) | Peak 2 | Peak 3 | Peak 4 | Peak 5 |
|---|---|---|---|---|
| 4 | 3.79 | 66.55 | 27.88 | 1.78 |
| 6 | 4.28 | 72.39 | 21.28 | 2.04 |
| 8 | 7.65 | 73.95 | 15.94 | 2.47 |
| 10 | 7.38 | 77.62 | 12.97 | 2.03 |
| 12 | 7.45 | 79.29 | 11.05 | 2.21 |
| 14 | 9.51 | 81.24 | 6.12 | 3.13 |
| 16 | 10.11 | 83.39 | 1.94 | 4.55 |

Preparation of ACH3 using a complexing or buffering agent. Samples of 8% Al (w/w) Reach™ 103 aluminum chlorohydrate are prepared with varied concentrations of glycine. The concentration of glycine is 10:1, 4:1, 2:1, 1:1, 1:1.25, and 1:1.5 Al to glycine molar ratio. Shortly after preparation, the samples are aged in 50° C. oven for 2 hours and subsequently analyzed using SEC-RI after diluting to 1% Al. 8% Al is chosen, because of the glycine solubility problems associated with higher concentrated ACH solutions. The highest amount of Peak 3 is observed for the sample with 1:1 Al to glycine molar ratio. While the data did not achieve a desired amount of Peak 3 under the conditions tested, the results for the Al to glycine ratio can be used under other conditions to increase the Peak 3 concentration.

TABLE 2
SEC Data of 8% Al Reach ™ 103 ACH
Aging Study (2 hrs. 50° C.)

| Sample Al:Gly | Gly/Al | Peak 2 | Peak 3 | Peak 4 | Peak 5 |
|---|---|---|---|---|---|
| 8% ACH | 0.00 | 8.28 | 73.37 | 15.94 | 2.47 |
| 10:1 Glycine | 0.10 | 4.00 | 75.71 | 16.63 | 3.66 |
| 4:1 Glycine | 0.25 | 3.26 | 77.53 | 14.15 | 5.05 |
| 2:1 Glycine | 0.50 | 3.14 | 78.00 | 11.74 | 7.13 |
| 1:1 Glycine | 1.00 | 3.00 | 79.66 | 9.59 | 7.75 |
| 1:1.25 Glycine | 1.25 | 2.74 | 79.50 | 9.19 | 8.57 |
| 1:1.5 Glycine | 1.50 | 2.56 | 79.28 | 9.45 | 8.71 |

The effect of more extended aging is then evaluated as follows: 8% Al (w/w) ACH 103 solutions are prepared and aged (55° C.) in presence of glycine at varying times:

TABLE 3
SEC Data of 8% Al Reach ™ 103 ACH w/Glycine Aging (55° C.)

| Time (hrs) | Peak 2 | Peak 3 | Peak 4 | Peak 5 | Peak 3:4 |
|---|---|---|---|---|---|
| 0 | 2.72 | 57.59 | 32.48 | 7.21 | 1.77 |
| 2 | 2.96 | 82.12 | 7.08 | 7.83 | 11.60 |
| 4 | 1.87 | 83.35 | 6.56 | 8.23 | 12.71 |
| 27 | 2.33 | 85.85 | 2.73 | 9.09 | 31.45 |
| 48 | 1.94 | 85.92 | 3.40 | 8.74 | 25.27 |
| 75 | 2.33 | 85.89 | 3.04 | 8.74 | 28.25 |
| 192 | 1.51 | 85.77 | 2.64 | 10.08 | 32.49 |

Aging 8% Al ACH 103 with 1:1 Al to glycine ratio for 27 hours at 55° C. is enough for Peak 2 and Peak 4 to reach a minimum, equilibrated concentration. The Peak 5 fraction can be further reduced by diluting the 8% Al ACH103 with 1:1 Al to glycine ratio (27 hrs 55° C.) to 4.8% Al and aging it further (90° C. for 35 mins) to provide a product with the SEC profile showing 94.75% of total peak area under peak 3.

²⁷Al NMR spectrum of this material shows a dominant peak at 11 ppm (octahedral Al) and a tiny peak at 70 ppm (tetrahedral Al). This data suggests a molecular structure of peak 3 Al species with essentially all aluminums octahedrally coordinated.

An alternative approach to synthesis of high Peak 3 material is to start with high Peak 4 material (Al30) and convert it.

Previously mentioned work synthesizes Peak 3 from Reach™ 103 aluminum chlorohydrate in appreciable purity. This synthesis pathway produces a polyaluminum chloride (PACl) solution with dominant peak 3 and undesired amounts of peak 5 (˜5%). In order to synthesize pure peak 3 with reduced peak 5, pure peak 4 (Al30) solution is used instead of ACH103.

Peak 3 solution is synthesized using pure peak 4 as starting material. Pure peak 4 solution (0.2% Al w/w) is freeze dried and reconstituted into 14.9% Al (w/w). This solution is then aged for 24-27 hours in a 50-55° C. oven. SEC chromatogram shows near pure peak 3, obtained by aging 14.9% Al peak 4 (Al30) solution for 24 hours at 50° C. By substituting the Al precursor, peak 5 in the final product is reduced to 2.5%.

### Example 2

### Flocculation Using High Peak 3 Aluminum Chlorohydrate

Aluminum AP active salts exhibit interesting properties desirable for removing colloids in waste water treatment plants, which is also relevant to the antiperspirant effect. Particles in waste water or sweat glands, usually negatively charged, fail to lump together due to electrostatic repulsions. Flocculating agents like ACH are added or diffuse into the aqueous solution to neutralize, agglomerate, and settle out the negatively charged colloids to purify water or block sweat glands. The current work evaluates the ability of a novel AP active salt, namely Peak 3, in comparison to commercial ACH to be used as flocculating agents for application in AP salts and waste water treatment agent. Supernatant turbidity is measured for waste water after treatment with aluminum AP active salts, particularly Peak 3 vs. commercial Aluminum Chlorohydrate (ACH).

Synthetic waste wafer is freshly prepared using toothpaste, fabric softener, liquid hand soap, and dish detergent. The prepared waste water exhibits extremely high turbidity (0.5% transmission), almost no transparency, and some undesired precipitate on the bottom. 400 mL of this water is carefully poured into 500 mL Erlenmeyer flasks so that precipitate was not transferred into samples.

Solutions of 3.75% Al (w/w) are prepared using ACH 103 powder and Peak 3 solution, prepared in accordance with Example land shown in Table 4.

TABLE 4
Concentrations of AP Actives

| | Amount | Amount | Solution | Al Conc. |
|---|---|---|---|---|
| Sample | Solution (g) | Powder (g) | Weight (g) | (%) |
| ACH 103 Powder | | 1.4829 | 9.9851 | 3.73 |
| Peak 3 Solution | 4.6878 | | 10.0044 | 3.75 |

Two 500 mL Erlenmeyer flasks are filled to 400 mL with the prepared synthetic waste water. Magnetic stir bars are added and flasks are placed on a 4-plate stirrer ensuring identical stirring conditions. Procedure is carried out in the following steps:
10 min at 500 RPM
10 min at 50 RPM
10 min settling.

Following addition of AP active, both samples separate into a clear top portion and white flocs on the bottom. Turbidity measurements of the clear top portion were made using a Turboscan™ LAB, and reported as percent transmission. The sample treated with Peak 3 solution has 89.8% transmission, while the commercial antiperspirant has 87.8%

After addition of 10 mL of 3.75% Al solutions into 400 mL of waste water and 12 hour settling period, the sample with ACH103 is more cloudy and less transparent than Peak 3 sample to the naked eye. Volume of floc is comparable between Peak 3 and ACH103. Turbidity measurements suggest Peak 3 removes suspended colloids more efficiently than ACH103. Supernatant transmission of Peak 3 sample is reported at 2% higher than its ACH103 counterpart. The result shows the Peak 3 material has somewhat better flocculating capability than the commercial ACH control.

### Example 3

### Use of High Peak 3 Aluminum Chlorohydrate in Oral Care

We have discovered that antiperspirant salts, mainly Zirconium Glycine (ZG) and Aluminum Chlorohydrates (ACH), relieve dentin hypersensitivity by chemically precipitating and physically occluding dentin tubules. The current invention, supported by hydraulic conductance experiments, provides Peak 3 Al species which is able to effectively precipitate in and occlude exposed dentin tubules for treatment of dentin hypersensitivity.

ACH3 material (95% Peak 3, 5% Peak 5, other peaks not seen by SEC-RI), is synthesized with glycine, in accordance with the previous example effectively reduces flow within exposed. dentin tubules via precipitation.

Human molars are cut into appropriately sized dentin disks. Disks are acid etched, for 35 seconds, in 6% citric acid to expose dentin tubules and then sonicated in DI for 30 minutes. Disks are placed in phosphate buffer solution (PBS) overnight with constant shaking. Using Flodec hydraulic conductance, a baseline flow rate is measured for 10 minutes using 400 μL PBS. The disks are treated for 2 minutes with 200 μL Peak 3 solution (4% Al w/w) treatment and 200 μL saliva. The dentin disks are rinsed twice with 400 μL fresh saliva. Procedure for control is identical, except for the treatment application (saliva application only). The flow rate through dentin tubules is measured after two successive treatments. Hydraulic conductance data demonstrates superb flow reduction within exposed dentin tubules. Flow reduction, reported as percentage from baseline, is set forth in Table 5.

TABLE 5
Dentin Tubule Occlusion with Peak 3

| | | Flow |
|---|---|---|
| Trial | Treatment | Reduction (%) |
| Control | No Treatment | 18 |
| 1 | Peak 3 (1:1 mol Gly) | 98 |
| 2 | Peak 3 (1:1 mol Gly) | 93 |
| Average Peak | | 95.5 |
| 3 Occlusion | | |

This hydraulic conductance data suggests that solution of predominantly Peak 3 material is a viable option for treating dentin hypersensitivity. This Al compound effectively blocks exposed dentin tubules, via precipitation, showing minimum 93% occlusion.

1. An aluminum chlorohydrate salt comprising a Peak 3:Peak 4 ratio of at least 10:1 and an amount of Peak 3 material relative based on a total of Peaks 2, 3, 4, and 5 is at least 80% as measured by size exclusion chromatography.

2. The salt of claim 1 wherein a Peak 3:Peak 4 ratio is at least 15:1.

3. The salt of claim 1, wherein a Peak 3:Peak 2 ratio is at least 10:1.

4. The salt of claim 1, wherein the amount of Peak 3 material relative based on the total of Peaks 2, 3, 4, and 5 is at least 90%.

5. The salt of claim 1, further comprising one or more complexing agents chosen from a) amino acids, b) ammonium acids, c) polyols, d) hydroxyl acids, e) carboxylic acids, and f) sulfonic acids.

6. The salt of claim 5, wherein the complexing agent comprises glycine.

7. The salt of claim 5, wherein the complexing agent is present in a complexing agent:aluminum molar ratio of not greater than 3:1.

8. The salt of claim 1, further comprising zirconium.

9. The salt of claim 1, which is substantially free of calcium ion.

10. The salt of claim 1, wherein the Peak 3:(Peak 4+Peak 2) ratio is at least 10:1.

11. The salt of claim 1, wherein Peak 3:Peak 4 ratio is at least 10:1 and an amount of Peak 3 material relative based on a total of Peaks 2, 3, 4, and 5 is at least 80% as measured by size exclusion chromatography when the salt is made by heating an initial aluminum salt solution, and wherein the aluminum salt is at least one of aluminum chloride, aluminum chlorohydrate, aluminum chlorohydrex polyethylene glycol, aluminum chlorohydrex propylene glycol, aluminum dichlorohydrate, aluminum dichlorohydrex polyethylene glycol, aluminum dichlorohydrex propylene glycol, aluminum sesquichlorohydrate, aluminum sesquichlorohydrex polyethylene glycol, aluminum sesquichlorohydrex propylene glycol, aluminum zirconium octachlorohydrate, aluminum zirconium or tachlorohydrex glycine, aluminum zirconium pentachlorohydrate, aluminum zirconium pemachlorohydrex glycine, aluminum zirconium tetrachlorohydrate, aluminum zirconium tehachlorohydrex glycine, aluminum zirconium trichlorohydrate, and aluminum zirconium trichlorohydrex glycine.

12. A composition comprising a salt according to claim 1 in combination or association with a substantially anhydrous carrier.

13. A water treatment composition comprising a salt according to claim 1.

14. An antiperspirant composition comprising a salt according to claim 1 and an antiperspirant carrier.

15. An oral care product comprising a salt according to claim 1 and an oral care carrier.

16. The oral care product of claim 15, wherein an amount of aluminum in the product is 3 to 20% by weight.

17. A method of making a salt according to claim 1, comprising heating an initial aluminum salt solution at a temperature of 40-80° C., optionally in presence of a complexing agent, until the Peak 3:(Peak 4+Peak 2) ratio is at least 10:1, wherein the aluminum salt is at least one of aluminum chloride, aluminum chlorohydrate, aluminum chlorohydrex polyethylene glycol, aluminum chlorohydrex propylene glycol, aluminum dichlorohydrate, aluminum dichlorohydrex polyethylene glycol, aluminum dichlorohydrex propylene glycol, aluminum sesquichlorohydrate, aluminum sesquichlorohydrex polyethylene glycol, aluminum sesquichlorohydrex propylene glycol, aluminum zirconium octachlorohydrate, aluminum zirconium octachlorohydrex glycine, aluminum zirconium pentachlorohydrate, aluminum zirconium pentachlorohydrex glycine, aluminum zirconium tetrachlorohydrate, aluminum zirconium tetrachlorohydrex glycine, aluminum zirconium trichlorohydrate, and aluminum zirconium trichlorohydrex glycine.

18. A method of treating water comprising adding the composition of claim 13 to water.

19. A method of reducing perspiration comprising applying the antiperspirant of claim 14 to skin.

20. A method of treating or reducing dental hypersensitivity and/or erosion comprising applying an effective amount of oral care product according to claim 15 to the teeth of a patient in need thereof.

21. (canceled)
